(19) United States
(12) Patent Application Publication
(10) Pub. No.: US 2023/0008313 A1
(43) Pub. Date: Jan. 12, 2023

(54) METHOD FOR FREEZING AND STORING NEONATAL STROMAL CELLS

(71) Applicant: VETBIOBANK, Marcy L'Etoile (FR)

(72) Inventors: Stéphane MADDENS, Roquefort la Bedoule (FR); Nathalie SAULNIER, Ecully (FR); Rodolphe RAKIC, Lyon (FR)

(21) Appl. No.: 17/790,269

(22) PCT Filed: Jan. 4, 2021
(86) PCT No.: PCT/FR2021/050005

§ 371 Date: Jun. 30, 2022

(30) Foreign Application Priority Data
Jan. 2, 2020 (FR) 20 00017

(51) Int. Cl. A01N 1/02; A61K 35/35

(52) U.S. Cl. CPC A01N 1/0284; A01N 1/0221; A61K 35/35

(57) ABSTRACT

The present invention relates to a method for freezing and preserving a composition comprising a population of neonatal stromal cells (NSCs) and a cryoprotector, characterised in that it comprises a step of freezing the composition at a temperature of between −70° C. and −140° C., then a step of preserving the composition at between −10° C. and −40° C. The present invention also relates to a composition comprising a population of NSCs and a cryoprotector, characterised in that it is preserved at between −10° C. and −40° C., said NSCs being in particular placental NSCs.

### TECHNICAL FIELD

The present invention relates to a method for freezing and preserving neonatal stromal cells (NSCs).

### PRIOR ART

Mesenchymatous stromal cells (MSCs) at the present time represent a cell therapy option in the context of degenerative and/or diseases involving a problem with the immune system. In 2019, MSCs are being used in more than 220 clinical trials in the course of recruitment on a worldwide scale and are generating keen interest from an industrial point of view.

The preservation capability of MSCs therefore represents a major technological lever for ensuring therapeutic efficacy of the end product. Technical developments during the past years have mainly been based on the chemical composition of the cryopreservation solutions and the freezing method for storing at ultra-low temperature. This work made it possible to achieve controlled freezing and long-term storage of MSCs in ultra-low temperature conditions between −80° C. and −195° C. (Marquez-Curtis et al., 2015). This range of temperatures involves having recourse to management of nitrogen for preservation in liquid or gaseous phase. An alternative is having recourse to high-performance freezers in order to maintain such temperatures. Such preservation therefore requires expensive equipment, logistics and access to a specialised storage site.

In 2012, Yingbo et al. (CN102487939 A) preserved, at −20° C. for a period of 20 days, a suspension of human placenta NSC in a freezing medium composed of glucose, sodium chloride, albumin, sodium bicarbonate, propylene glycol and cholesterol. The freezing method used is freezing at −20° C. for storage at between −20° C. and 4° C. The authors demonstrated viability on thawing of the order of 85%.

In 2015, the team of Espina et al. froze bone marrow MSCs at −150° C. and subjected them to low-temperature conditions mimicking delivery conditions. The team showed that it was difficult to envisage preservation of the bone marrow MSCs at temperatures of −20° C. for a period of 48 h to 72 h at a maximum. These results show at a minimum a drop in viability of around 50% of the cells on thawing.

There was therefore a need for preserving MSCs by means of less expensive devices that were easy to use, by the doctor or veterinary surgeon for example. It was also necessary to develop a preservation method that guarantees acceptable viability of the cells after thawing and the maintenance of therapeutic efficacy.

The inventors unexpectedly discovered that a method for freezing and preserving a composition comprising a population of neonatal stromal cells (NSCs) and a cryoprotector, comprising a step of freezing at between −70° C. and −140° C. followed by a step of preservation at between −10° C. and −40° C., made it possible to preserve the NSCs at a temperature of between −10° C. and −40° C. over a long period (in particular at least three weeks, or even several months), while guaranteeing acceptable cell viability, metabolic activity and immunomodulation capability after thawing.

This possibility of preserving NSCs at a temperature of between −10° C. and −40° C. while keeping good viability is all the more surprising since the prior art reported a significant loss of viability during preservation at such temperatures (Freshney R. (2015), Mazur, P. (1984), Gao et al. (1997)).

Thus, the inventors have developed a method allowing preservation of NSCs by means of low-temperature preservation devices at between −10° C. and −40° C., easy to implement and inexpensive, such as standard freezers for non-industrial use, and guaranteeing good viability of the cells and therapeutic efficacy thereof after several months of preservation.

### DESCRIPTION OF THE INVENTION

The present invention relates to a method for freezing and preserving a population of neonatal stromal cells.

Within the meaning of the present invention, “population of cells” means a set of cells comprising one or more types of different cells, for example a mixture of cells at various stages of differentiation, comprising cells having the same tissue origin or coming from different tissue origins.

From a structural point of view, neonatal stromal cells (NSCs) can be characterised by the fact that fewer than 10% of the cells express one or more of the following surface markers: CD11b, CD14, CD31, CD34, CD45 or HLA-DR, also referred to as MHC-II. NSCs can also be characterised by the expression at the transcriptional level of growth factors, such as the vascular endothelium growth factor (VEGF), the growth factor (HGF), the keratinocyte growth factor (KGF), or the transforming growth factor β (TGF-β).

The NSCs according to the invention can also be characterised by their biological properties, such as their capacity for cell proliferation.

Thus, in a specific embodiment, the population of NSCs is characterised in that at least 80% of the cells of said population of NSCs possess a capacity for consecutive cell doubling greater than 20 total doublings.

“Capacity for cell proliferation” means that a population of NSCs according to the invention is capable of doubling in number, more than 20 times in total.

To evaluate the number of doublings, at each cell passage, the cells are detached from their support, centrifuged and then counted, for example by the Trypan Blue exclusion technique using an electronic counter. The number of doublings at each cell passage is calculated according to the following formula: number of doublings=LOG (Nf/Ni)/LOG(2) (Nf: number of final cells and Ni: number of initial cells). The total number of cell doublings is equal to the sum of the number of total doublings at each cell passage.

The population of NSCs can also be characterised in that at least 80% of the cells of said population of NSCs have:

a capacity for adhesion to a plastic support; and/or

a chondrogenic differentiation potential, and/or

an immunomodulator potential.

In a specific embodiment, the population of NSCs is also characterised in that at least 80% of the cells of said population of NSCs have a limited osteogenic differentiation potential or do not have any osteogenic differentiation potential.

“Capacity for adhesion to a plastic support” means that the population of NSCs is characterised by its property of adhesion to a plastic support.

“Chondrogenic differentiation potential” means that the NSCs have the capability of being able to differentiate into chondrocytes. The expressions “chondrocyte differentiation” and “differentiation into chondrocytes” can also be used indifferently.

This capacity for differentiation into chondrocytes can be evaluated by the protein and/or transcriptional study of the specific markers of cartilage such as type II collagen (COL2A1), SOX-9 (SOX9), aggrecan (ACAN), cartilage oligomeric matrix protein (COMP), type IX collagen (COL9A1), type XI collagen (COL11A1), type IIB collagen (COL2B) after induction of chondrogenesis. Moreover, viscoelastic properties of the extracellular matrix, expressed by the differentiated NSCs, are similar to the viscoelastic properties of cartilage. These viscoelastic properties can thus serve as a characteristic for evaluating chondrogenesis.

In a favoured culture mode, to induce chondrogenesis, the NSCs are detached from their support by trypsination and used for forming micromasses by gravitation in a drop of amplification medium such as for example DMEM (1 g/L of glucose) supplemented with 10% SVF (vol:vol), 2 mM of glutamine and 0 to 20 ng/ml of fibroblastic growth factor (FGF2-β). A micromass of NSCs then forms after 24 h of incubation, at the base of the drop. This micromass is recovered and put in a chondrocyte differentiation medium composed of DMEM with 4.5 g/L of glucose and with TGF-β3 or a TGF-β1/BMP-2 combination added, for 7 to 28 days. At the end, an RNA or protein extraction is implemented in order to analyse the expression of specific markers of the chondrogenic lineage such as type II collagen, aggrecan, COMP or SOX9.

“Immunomodulator potential” means the capacity for immunomodulation of the NSCs.

This potential can be characterised by the capacity that the NSCs have for expressing a set of immunomodulator factors such as PGE2, IL-6, IL-10, TGF-β, IDO, iNOS, HGF, KGF, CCL2 and/or TSG-6. This is because, in the presence of an inflammatory context, NSCs can modify their phenotype. The inflammatory context can be mimicked in vitro by stimulation of the cells by means of cytokines and/or growth factors such as IFN-γ, IL-1, IL-6 and/or TNF-α. A modification of the phenotype of the NSCs results in the ability of the NSCs to modify the transcription and/or protein expression of markers involved in the immunomodulation. Among these markers mention can be made of PGE2, IL-6, IL-10, TGF-β, IDO, iNOS, HGF, KGF, TSG-6, CCL2, CMH-I, CMH-II, HLA-E. Thus, the immunomodulator potential can for example be determined by studying the expression of prostaglandin (PGE2) secreted by cells in basal condition and after stimulation in an inflammatory context. For this purpose, the cells are cultivated in a proliferation medium (medium with foetal bovine serum) or in a medium complemented with gamma interferon, and then the secreted PGE2 is measured by ELISA test.

The immunomodulator potential of NSCs can also be characterised by the antiproliferative effect of the NSCs on peripheral blood mononucleated cells (PBMCs) treated with a mitogenic agent such as phytohaemagglutinin, concanavalin A and/or lipopolysaccharides. Immunomodulation of the NSCs can also be evaluated by the ability of the NSCs to inhibit the proliferation, the secretion of pro-inflammatory cytokines and/or the differentiation of T or NK lymphocytes, B lymphocytes, monocytes and/or macrophages.

Thus, the immunomodulator potential of NSCs can also be determined by the ability of the NSCs to inhibit the proliferation of lymphocytes in vitro. For this purpose, blood mononucleated cells (PBMCs) previously incubated with a fluorescent dye are co-cultivated with the NSCs in an NSC/PBMC proportion of 1:10 in the presence of a mitogenic agent such as concanavalin A. After 4 days of culture at 37° C., the non-adherent cells are recovered and marked by an anti-CD3 antibody (specific to T lymphocytes) coupled to a fluorochrome, and then analysed in flow cytometry to evaluate the signal of the fluorescent dye within the positive CD3 population. The comparison of the marking with that achieved with a control corresponding to the PBMCs cultivated in the presence of a mitogenic agent but without NSC (PBMC-ctrl), makes it possible to define a proliferation index for the lymphocyte population. With a (CSN+ PBMC 1:10)/(PBMC-ctrl) ratio of less than or equal to 0.5, the NSCs are considered to be exerting a significant antiproliferative activity (FIG. 7 and FIG. 8).

The population of NSCs can also be characterised in that at least 80% of the cells of said NSC population do not have an osteogenic differentiation potential. A low osteogenic differentiation potential or low osteogenesis potential is also spoken of.

This absence of an osteogenic differentiation potential or this low osteogenic differentiation potential can be evaluated by the protein and/or transcriptional study of the specific markers of bone (ALPL, RUNX2, etc.) or by analysis of the calcic deposits after induction of osteogenesis in 7 to 15 days. This osteogenic induction can be achieved by culture of the NSCs in monolayer in the presence of an osteogenic differentiation medium containing a corticosteroid, such as dexamethasone (0.1-1 μM), a reducing agent such as ascorbic acid 2-phosphate (between 0 and 200 μg/ml) and β-glycerophosphate (0-50 mM). BMP-2 may for example replace dexamethasone.

At the end of the differentiation process, the presence of calcic deposits in the culture dish is revealed by colouring with a 1% solution of alizarin red (weight/volume) under microscope. The absence of deposits reveals the absence of osteogenic differentiation potential while the presence of calcic deposits coloured by an intense red shows the presence of an osteogenic differentiation.

Alternatively or in addition, a protein and/or transcriptional study of the specific markers of bone (ALPL, RUNX2) is implemented.

This characteristic thus enables the NSCs to be used in cell therapy. In particular, it is a case of cell therapy for the treatment of arthropathy, since they limit the generation of calcified ectopic tissue in the joint. This property is particular important for applications such as arthrosis, with or without tissue damage.

According to a first aspect the present invention relates to a method for freezing and preserving a composition comprising a population of NSCs and a cryoprotector, characterised in that:
1) said composition is frozen at a temperature of between −70° C. and −140° C., in particular between −70° C. and −100° C., more particularly at −80° C.; then
2) said composition is preserved at between −10° C. and −40° C., in particular between −10° C. and −35° C., more particularly between −15° C. and −30° C., even more particularly between −17° C. and −20° C., even more particularly at −18° C.

The inventors discovered surprisingly that such a method made it possible to guarantee cell viability and acceptable metabolic activity as well as immunomodulating activity of the NSCs, when they are preserved at a temperature between −10° C. and −40° C. The immunomodulating activity of the NSCs can for example be assessed through their immunosuppressive activity, in particular their ability to inhibit the proliferation of lymphocytes in vitro.

In particular, step 1 is implemented for example by a temperature drop to a temperature of between −70° C. and −140° C., in particular between −70° C. and −100° C., more particularly to −80° C., at the rate of −1° C./minute.

In a particular embodiment, step 1 is of at least 2 h. In a more particular embodiment, the cells are preserved at a temperature of between −70° C. and −140° C., in particular between −70° C. and −100° C., more particularly at −80° C., at step 1 between 2 h and 36 months, more particularly between 2 h and 24 months.

Optionally, an intermediate step of storage of the composition at a temperature below −70° C., in particular between −70° C. and −196° C., between step 1 and step 2, can be implemented. In particular, this intermediate storage is for 1 day to 24 months, in particular 1 day to 24 months, for example 2 weeks to 3 months.

Step 2, referred to as the storage step, corresponds to the preservation of the composition comprising the NSCs and a cryoprotector, at a temperature between −10° C. and −40° C., in particular between −10° C. and −35° C., more particularly between −15° C. and −30° C., even more particularly between −17° C. and −20° C., even more particularly at −18° C.

In a particular embodiment, the preservation of the composition at step 2 is from 1 day to 5 months, in particular 1 day to 4 months, more particularly 1 day to 3 months.

In a particular embodiment, the preservation of the composition at step 2 is from at least 21 days, in particular between 21 days and 5 months, more particularly between 21 days and 3 months.

It should be noted that said composition can be packaged in unit doses in order to constitute injectable solutions ready for use. The number of NSCs in these unit doses is defined later in the application.

“Cryoprotector” means any compounds making it possible to provide the cryopreservation and freezing function. A suitable cryoprotector according to the invention is capable of guaranteeing the integrity and formulation of the composition.

“Integrity of the formulation of the pharmaceutical composition” means that the structural characteristics of the NSCs, the biological characteristics of the NSCs and the proportion of NSC in the pharmaceutical composition are not lost or drastically modified. More particularly, the parameters mentioned previously relate to the adhesion capability of the NSCs, the capacity for proliferation of the NSCs, the expression of the phenotype markers previously mentioned, the capacity for differentiation of the NSCs, the capacity for immunomodulation of the NSCs, the cell concentration of the pharmaceutical composition and/or the cell viability. More particularly, the capacity for immunomodulation of the NSCs and more broadly the pharmaceutical composition are concerned.

A pharmaceutical composition wherein 50% to 99% of the NSCs initially used for formulating the composition are capable of adhering to the plastic and proliferating, more particularly 70 to 99%, more particularly 80% to 99%, is considered to be not drastically modified.

A pharmaceutical composition wherein the concentration of the NSCs represents 50% to 99% of the initial concentration of NSCs used for formulating the composition, more particularly 70 to 99%, more particularly 80% to 99%, is considered to be not drastically modified.

A pharmaceutical composition wherein the proportion of viable NSCs is greater than 50% of the viable cells initially present in the pharmaceutical composition, more particularly greater than 70%, more particularly greater than 80%, is considered to be not drastically modified.

A pharmaceutical composition wherein the NSCs expressing the markers normally expressed by the NSCs at expression percentages above 60%, more particularly 70%, more particularly 80% among the total population of NSCs, is considered to be not drastically modified.

A pharmaceutical composition including NSCs keeping an inhibition activity on the proliferation of PBMCs as described in said invention, and this for a number ratio of 1 NSC for 10 PBMCs, is considered to be not drastically modified. More particularly, this inhibition activity can be evaluated through the ratio between the proliferation of the PBMCs stimulated by a mitogenic agent in the presence of the NSCs and the proliferation of the PBMCs stimulated by a mitogenic agent alone. This concept is defined in the invention by “PBMC proliferation ratio”. This inhibition activity is expressed by a reduction in the PBMC proliferation ratio greater than 20% of the proliferation activity of the PBMCs stimulated by a mitogenic agent, more particularly greater than 30%, more particularly greater than 50%.

According to one embodiment, said cryoprotector is chosen from dimethyl sulfoxide (DMSO), glycerol, propylene glycol, proteoglycans, trehalose, “bovine serum albumin” (BSA), synthesis of extraction albumin, plasma enriched to a greater or lesser extent with platelets or platelet content, gelatin, polyethylene glycol (PEG), polyacrylic acid, poly-L-lysine, ethylene glycol or a combination of a plurality of these cryoprotectors.

According to one embodiment, said cryoprotector is selected from dimethyl sulfoxide (DMSO), glycerol, propylene glycol, proteoglycans, trehalose, gelatin, polyethylene glycol (PEG), polyacrylic acid, poly-L-lysine, ethylene glycol or a combination of a plurality of these cryoprotectors.

In a particular embodiment, said composition comprises between 0.5 and 30%, in particular 0.5 and 20%, in particular 2 and 10%, more particularly 5% cryoprotector.

In a particular embodiment, said composition comprises DMSO as cryoprotector. In a particular embodiment, said composition comprises from 0.5 to 30% DMSO, more particularly 2 to 10% DMSO, even more particularly 5% DMSO.

In a particular embodiment, said composition comprises glycerol as cryoprotector. More particularly, said composition comprises 2 to 20% glycerol.

In a particular embodiment, said composition comprises propylene glycol or poly-L-lysine as cryoprotector.

Commercial solutions comprising a cryoprotector can also be used in said composition, such as STEMALPHA.CRYO3 (StemAlpha, France), CryoStor® CS2, CS5 or CS10 (STEMCELL Technologies, France) solutions.

In a particular embodiment, said composition may furthermore comprise between 1 and 20% foetal bovine serum (FCS), more particularly between 5 and 20%, even more particularly 10%.

In a particularly embodiment, said composition furthermore comprises D-PBS (Dulbecco's Phosphate-Buffered Saline), DMEM (Dulbecco's Modified Eagle Medium), or MEM (Minimum Essential Media).

In a particular embodiment, the composition may furthermore comprise DMEM comprising SVF, in particular 5 to 20% SVF and more particularly 10%.

In a particular embodiment, the composition may furthermore comprise one or more adjuvants such as ammonium chloride, Ringer's lactate or BSA.

In a particular embodiment, said composition is free from product of animal origin.

According to one embodiment, the population of NSCs in the composition comes from a neonatal tissue sample, in particular from one or more placentas and/or from one or more umbilical cords, and/or from one or more amniotic membranes, or from a sample of neonatal fluid, in particular the blood of one or more umbilical cords, or the amniotic liquid of one or more amniotic fluids.

The advantage of neonatal fluids makes it possible to have available a large number of NSCs from a sample and to provide certain structural characteristics.

For this, the extraembryonic annexes (placenta, umbilical cord, amniotic membrane) are generally sampled aseptically during caesareans or natural deliveries in gestating females, preferably at full term. For example, as soon as the newborn has emerged from the amniotic sac and has been made safe, the extraembryonic tissue is immediately transferred into a transport box containing for example a saline solution buffered with Dulbecco's phosphate to be conveyed to the laboratory. The umbilical cord blood for its part can be recovered by puncturing at the umbilical vein, in particular using a needle connected to a blood sampling pouch or a tube or any other container. In another example, the amniotic liquid can be recovered by puncturing through the amniotic membrane, in particular using a needle connected to a blood sampling pouch or a tube or any other container.

In a particular embodiment, said composition comprises a population of placental NSCs.

In a particular embodiment, the population of NSCs of the composition comes from neonatal mammal tissues or fluids, and in particular from a dog, a cat, a horse or a human.

In a particular embodiment, the neonatal tissues and fluids come from a dog, horse or cat.

In an even more particular embodiment, the population of NSCs of the composition is a population of dog placental NSCs.

The inventors have shown unexpectedly that the freezing method described in said invention, applied to this so-called MHC-I^{L}/CD90^{H} NSC population, allowed medium-term storage (from 3 weeks to several months) of the pharmaceutical composition without drastic modification to the properties of said pharmaceutical composition.

In a particular embodiment, the NSC population of the composition comprises NSCs of weak MHC-1 phenotype (MHC-I^{L}) and of strong CD90 phenotype (CD90^{H}).

In particular, said population of NSCs of the composition is characterised in that at least 80% by number, in particular at least 85%, more particularly at least 90% of the cells of said population are MHC-I^{L} and characterised in that at least 80% by number, in particular at least 85%, more particularly at least 90% of the cells of said population are CD90^{H}.

In a particular embodiment, said composition comprises a population of NSCs of MHC-1^{L}/CD90^{H} phenotype.

In a particular embodiment, said composition is free from NSCs of MHC-I^{H}/CD90^{L} phenotype.

MHC-I corresponds to the class 1 major histocompatibility complex, expressed almost ubiquitously in the organism.

MHC-1, also called HLA-1 (“human leukocyte antigen class 1”) in humans results from the expression of a family of genes called HLA genes. Among these genes mention can be made of HLA-A, HLA-B and HLA-C, which encode the conventional forms of HLA-I. In dogs, MHC is also called DLA (dog leukocyte antigen), in horses ELA (equine leukocyte antigen) is spoken of and in cats FLA (feline leukocyte antigen) is spoken of.

CD90 corresponds to the membrane protein “Thy-1 cell surface antigen” and is expressed by a large majority of stroma cells.

“MHC-I^{L} phenotype” means that the NSCs have a very weak expression or an absence of expression of MHC-1. “CD90^{H} phenotype” means that the NSCs have a strong expression of the CH90 surface antigen.

Conversely, “MHC-I^{H} phenotype” means that the NSCs have a very strong expression. “CD90^{L} phenotype” means that the NSCs have a weak expression of the CD90 surface antigen.

The inventors unexpectedly identified the fact that the MHC-I^{L}/CD90^{H} phenotype of the NSCs was related to the proliferation capability of the cells, enabling the latter to double a large number of times in vitro than the NSCs of phenotype MHC-I^{H}/CD90^{L}.

The NSCs of phenotype MHC-I^{L}/CD90^{H} also have a good immunomodulation capability.

In particular, said population of NSCs of the composition is characterised in that less than 20% by number, in particular less than 15%, more particularly less than 10% of the cells of said population are MHC-I^{H} and characterised in that at least 20% by number, in particular at least 15%, more particularly at least 10% of the cells of said population are CD90^{L}.

In a particular embodiment, the population of NSCs of the composition is free from NSC of phenotype strong MHC-I (CMH-I^{H}) and of phenotype weak CD90 (CD90^{L}).

The population of MHC-I^{L}/CD90^{H} NSCs has a particular ability to withstand freezing.

The present invention also relates to a pharmaceutical composition or an injectable solution ready for use comprising a population of neonatal stromal cells (NSCs), and a cryoprotector obtained by the method for freezing and preserving NSCs described above, preserved at between −10° C. and −40° C., in particular between −10° C. and −35° C., more particularly between −15° C. and −30° C., even more particularly between −17° C. and −20° C., even more particularly at −18° C. In particular, this pharmaceutical composition or injectable solution is preserved at these temperatures for at least 21 days, more particularly between 21 days and 5 months, typically between 21 days and 3 months.

The present invention also relates to a pharmaceutical composition or an injectable solution ready for use comprising a population of neonatal stromal cells (NSCs) and a cryoprotector, characterised in that it is preserved at between −10° C. and −40° C., in particular between −10° C. and −35° C., more particularly between −15° C. and −30° C., even more particularly between −17° C. and −20° C., even more particularly at −18° C.

In particular, this pharmaceutical composition or injectable solution is preserved at these temperatures for at least 21 days, more particularly between 21 days and 5 months, typically between 21 days and 3 months.

“Ready for use” means that the composition or injectable solution comprising a population of NSCs is ready to be injected into the individual with or without a step of transfer into an administration device (pouch, solute, syringe). Putting the cells back in culture before use in the individual is not necessary and cells do not need to be washed or put back in suspension in a physiological medium, and this even when they are formulated with a cryoprotector as described below. The expression “ready for use” signifies that only one thawing step is necessary when the composition or injectable solution is in frozen form, before injection into the subject.

Since this pharmaceutical composition or injectable solution can be frozen, it is thus usable at all times. This has the advantage of making the treatment available in the shortest possible time while limiting the human action necessary for its efficacy and limiting the risk of contamination inherent in each manipulation by an operator. Thus, it is possible to separate the method for obtaining the pharmaceutical composition from the final clinical use thereof.

The cryoprotector may be a cryoprotector as defined previously. Likewise, the quantities of cryoprotector in the composition are as described above.

In a particular embodiment, the cryoprotector is DMSO.

According to one embodiment, the population of NSCs of the pharmaceutical composition or of the injectable solution ready for use comes from a neonatal tissue sample, in particular from one or more placentas and/or from one or more umbilical cords, and/or from one or more amniotic membranes, or from a sample of neonatal fluid, in particular the blood from one or more umbilical cords, or the amniotic liquid from one or more amniotic fluids.

In a particular embodiment, the NSCs are placental NSCs.

In a particular embodiment, said pharmaceutical composition or injectable solution read for use comprises a population of NSCs and a cryoprotector, characterised in that it is preserved at between −10° C. and −40° C., in particular between −10° C. and −35° C., more particularly between −15° C. and −30° C., even more particularly between −17° C. and −20° C., even more particularly at −18° C., and in that said NSCs are placental NSCs.

In a particular embodiment, the population of NSCs of the pharmaceutical composition or of the injectable solution ready for use comes from neonatal tissues or fluids of mammals, in particular from dogs, cats, horses or humans.

In a more particular embodiment, the neonatal tissues or fluids come from a dog, a horse or a cat.

In an even more particular embodiment, the population of NSCs of the pharmaceutical composition or of the injectable solution ready for use is a population of dog placental NSCs.

In a particular embodiment, the population of NSCs of the pharmaceutical composition or of the injectable solution ready for use comprises NSCs of weak MHC-1 phenotype (MHC-I^{L}) and of strong CD90 phenotype (CD90^{H}).

In particular, said population of NSCs of the pharmaceutical composition or of the injectable solution is characterised in that at least 80% by number, in particular at least 85%, more particularly at least 90% of the cells of said population are MHC-I^{L} and characterised in that at least 80% by number, in particular at least 85%, more particularly at least 90% of the cells of said population are CD90^{H}.

The cells of phenotype MHC-I^{L}/CD90^{H} can be selected in flow cytometry by simultaneous marking (also referred to as immunophenotype double marking or co-marking) of the MHC-I and of the CD90 within the same population of NSCs.

This double marking can be implemented using:
for marking of MHC-I, a primary anti-mouse MHC-I IgG2a antibody, such as the primary anti-MHC-I DG-BOV2001/DG-H58A IgG2a antibody (Monoclonal Antibody Center Washington State University), revealed with a secondary goat F(ab′)2 secondary anti-mouse IgG antibody coupled to allophycocyanin (APC), and
for marking CD90, an anti-CD90 monoclonal antibody coupled to phycoerythrin (PE), such as the monoclonal anti-rat CD90/Thy1 antibody Antibody YKIX337.217 (PE).

After marking, the fluorescence of the APC and PE fluorochromes is analysed. In order to discern the sub-populations of NSC, a 2D representation of the APC and PE fluorescence is produced. The results are compared with those obtained with the use of the isotypes coupled to the respective fluorochromes described previously.

A population or sub-population of NSC is termed MHC-I^{L}/CD90^{H} if the ratio of the median fluorescence intensity (MFI) between the MHC-I and its control isotype (also referred to as relative MFI or rMFI) is below a threshold of 20, more particularly 15, more particularly 10, and if the rMFI between the CD90 marker and its control isotype is above 50, more particularly 20. On the other hand, a population or sub-population of NSC is termed MHC-I^{H}/CD90^{L} if the ratio of the median fluorescence intensity (MFI) between the MHC-I and its control isotype is above 20, more particularly 15, more particularly 10, and if the rMFI between the CD90 marker and its control isotype is below 15, more particularly 20. With double marking, the presence of various NSC sub-population profiles with regard to the expression of the MHC-I/CD90 markers (MHC-I^{L}, MHC-I^{H} and CD90^{H}, CD90^{L}) is revealed, in the same cell population coming from the same sample (FIG. 12 and FIG. 13).

In particular, selection by double marking is implemented after an amplification step comprising 3 to 5 successive cell passages.

In a particular embodiment, the NSC population of the pharmaceutical composition or of the injectable solution ready for use is free from NSC of CMH-I^{H}/CD90^{L} phenotype.

Alternatively, the cells of MHC-I^{H}/CD90^{L} phenotype can be excluded by negative selection, i.e. by not selecting in flow cytometry, as described above, the populations comprising at least 20% by number, in particular at least 15%, more particularly at least 10% of the MHC-I^{H}/CD90^{L} cells. In a particular embodiment of the present invention, said population of |NSCs of the composition or of the injectable solution ready for use is characterised in that at least 80% by number, in particular at least 85%, more particularly at least 90% of the cells of said population are MHC-I^{L} and characterised in that at least 80% by number, in particular at least 85%, more particularly at least 90% of the cells of said population are CD90^{H}.

In a particular embodiment of the present invention, said NSC population of the composition or of the injectable solution ready for use is characterised in that less than 20% by number, in particular less than 15%, more particularly less than 10% of the cells of said population are MHC-I^{H} and characterised in that at least 20% by number, in particular at least 15%, more particularly at least 10% of the cells of said population are CD90^{L}. In a particular embodiment, the NSC population of the composition or of the injectable solution ready for use is free from NSC of strong MHC-I (MHC-I^{H}) phenotype and of weak CD90 (CD90^{L}).

Typically, the pharmaceutical composition or the injectable solution includes an NSC population of 1×10⁶ to 1×10⁸ cells with a volume of 0.1 ml to 15 ml, i.e. a concentration of 5×10⁴ to 1×10⁹ cells/ml. In particular, it comprises from 1×10⁶ to 5×10⁷ cells in a volume of 0.1 ml to 15 ml, i.e. a concentration of 7×10⁴ to 5×10⁷ cells/ml. In particular, it comprises from 2.5×10⁶ to 1×10⁷ cells for a volume of 0.1 ml to 15 ml of composition, i.e. a concentration of 1.5×10⁵ and 1×10⁸ cells/ml. In particular, it comprises from 1×10⁶ to 1×10⁷ cells in 0.5 to 2 ml, i.e. a concentration of 5×10⁵ to 2×10⁷ cell/ml.

Typically, said composition or injectable solution comprises between 1×10⁶ and 1×10⁸ cells, in particular 1×10⁷ cells.

Another aspect of the present disclosure relates to a composition or an injectable solution as defined previously for therapeutic use thereof.

Typically, it is a case of use in cell therapy. “Cell therapy” means a therapeutic treatment comprising the administration of cells able to induce a beneficial therapeutic effect in the individual. In the context of a regenerative medicine approach, this cell therapy is able to directly (cell differentiation) or indirectly (secretion of biological factors, activation or inhibition of cells of the environment) favour the in vivo regeneration of one or more biological tissues in an individual awaiting such treatment. Alternatively, the cells, in particular NSC that is the subject of the administration, may or may not have undergone modification of the genetic type by means of homologous recombination tools or recombinations by programmable nucleases such as the CRISPR-Cas9 (“Clustered Regularly Interspaced Short Palindromic Repeats”) systems.

In particular, it is a case of a use in one and the same individual, or in an individual of the same species, or in an individual of a different species with respect to the species from which said NSCs come.

When the receiving subject is identical to the individual from whom the NSCs come, autologous therapeutic use is spoken of. When the receiving subject is an individual of the same species as the NSC provenance species, heterologous or allogenic therapeutic use is spoken of. When the receiving subject is an individual from a different species with respect to the NSC provenance species, xenogenic therapeutic use is spoken of.

In a particular embodiment, the present invention relates to a composition or an injectable solution as previously defined for xenogenic therapeutic use thereof.

In a particular embodiment, the present disclosure relates to said pharmaceutical composition or injectable solution as defined previously for therapeutic use thereof in a mammal. In particular, said mammal is a dog, a cat, a horse or a human.

By way of example, the therapeutic use may be the treatment of:
tissue or joint damage, with or without inflammatory component;
degenerative illnesses, in particular arthrosis, tendinopathies, tissue fibroses, Alzheimer's, Parkinson's;
auto-immune, inflammatory and/or infectious diseases, in particular atopic dermatitis, gingivostomatitis, thrombocytopenia, epidermolysis bullosa, sepsis, chronic inflammatory bowel diseases (CIBD);
graft rejection, or
tumoral diseases.

Tissue damage means lesions, loss of normal function and/or degradations caused by excessive stress on the tissue, normal stress on a pathological tissue or all tissues requiring tissue reconstruction/healing: myocardial infarction, renal damage, liver damage, burn, skin lesions, fractures, respiratory diseases, osteoarticular diseases such as osteochondritis dissecans.

Degenerative illness means all types of diseases where the homeostatic balance is disturbed in favour of exacerbated tissue catabolism or excessive induction of tissue anabolism, such as for example arthrosis, tendinopathies, tissue fibroses, Alzheimer's or Parkinson's.

The composition or the injectable solution defined previously can also be used in the treatment of physiological illnesses or irregularities related to undesired immune response, i.e. all types of diseases where the immune system interferes with the normal/physiological functioning of the tissue or with treatment aimed at treating an immune disease and/or resorbing the normal/physiological functioning of a tissue, such as for example atopic dermatitis or gingivostomatitis.

They can be used in the treatment of auto-immune and inflammatory diseases such as the reaction of a graft against the host (graft versus host disease or GvHD), a tissue or organ graft, auto-immune diseases such as plate sclerosis, tissue inflammation, allergy, asthma, allergic bronchitis, chronic bronchitis such as chronic obstructive pulmonary bronchopathy (COPB), chronic inflammatory bowel disease, renal failure, thrombocytopenia, lupus erythematosus, rheumatoid arthritis or epidermolysis bullosa.

“Chronic inflammatory bowel diseases (CIBD)” means idiopathic chronic inflammations of the mucous membrane of the small intestine, of the colon and of the anoperineal region, such as duodenal enteritis in cats and dogs, and more particularly inflammations such as Crohn's disease and haemorrhagic rectocolitis in humans. These illnesses are characterised by gastrointestinal and chronic disorders associated with inflammatory infiltration of the mucous membrane. They are usually diagnosed when there is vomiting and chronic diarrhoea in the animal and in the human. CIBDs are distinct from enteropathies responding to a change in food and diarrhoeas responding to antibiotics. By definition, they respond to immunosuppressors rather than to specific food or to antibiotics. The clinical signs such as vomiting, diarrhoea, weight loss or loss of appetite are due to cellular infiltrates of the mucous membrane, to inflammation mediators, to malfunctioning of the enterocytes associated with inflammation and disorder of the motility of intestine.

The composition or the injectable solution defined previously can also be used in the treatment of diseases in which chronic inflammation related or not to immune disorder causes tissue degenerescence or malfunctioning of the functioning of an organ or of a tissue such as arthrosis or tendinitis.

Moreover, the composition or the injectable solution defined previously can be used in the treatment of infectious diseases associated or not with the preceding components. Infectious diseases means diseases involving contamination by pathogens such as microorganisms such as protozoa, bacteria and/or viruses. These diseases with an infectious component may be of a localised or systemic nature. The use of NSCs may in particular be prescribed in the particular context of resistance of the pathogens involved to antibiotics and in the context of a generalised inflammatory response associated with a serious infection, such as sepsis or septicaemia. By way of example, sepsis may be caused by a pathogen causing meningitis (Purpura fulminans), a pathogenic bacterium, a non-pathogenic bacterium, a virus of the SARS type (SARSr-CoV), a virus of the influenza type or a virus causing haemorrhagic fever.

The therapeutic context may extend to illnesses having several pathological aspects such as arthrosis with a degenerative aspect and an inflammatory aspect.

The previously defined composition or injectable solution may also be used in the treatment of illnesses of a tumoral nature with or without a metastasic component.

The therapeutic context may also extend to the use of the NSCs combined with other types of therapies such as for example: laser; shock waves, platelet-rich plasma (PRP); hyaluronic acid; non-steroid anti-inflammatories.

In a particular embodiment, the present disclosure relates to the previously defined composition or injectable solution for use thereof in tissue engineering. “Tissue engineering” means all biotechnology techniques using cells and biomaterials (of biological or synthetic origin) for generating tissue substitutes in vitro/ex vivo for in vivo implantation or for being used as a tissue model in the laboratory (for example: skin, bone or cartilage reconstruction).

In a particular embodiment, the present disclosure relates to the previously defined composition or injectable solution for use thereof in the treatment of indications of the musculoskeletal system such as arthrosis in mammals, more particularly in dogs, cats or horses, preferably in dogs.

In another particular embodiment, the present disclosure relates to the previously defined composition or injectable solution for use thereof in the treatment of thrombocytopenia in mammals, more particularly in dogs, cats or horses, preferably in dogs.

In a particular embodiment, the present disclosure relates to the previously defined composition or injectable solution for use thereof in the treatment of chronic inflammatory bowel disease, such as duodenal enteritis in mammals, more particularly in dogs, cats or horses, preferably in dogs.

In a particular embodiment, the present disclosure relates to the previously defined composition or injectable solution for use thereof in the treatment of chronic inflammatory bowel disease, such as Crohn's disease in humans.

The composition or injectable solution as previously defined can be administered externally topically, locally, i.e. by intra-articular, intramuscular, subcutaneous or intraspinal route) or intravenously (IV), intra-arterially or more generally by parenteral route.

Typically, a local administration may be an administration by intra-articular injection, for example in the case of the treatment of indications of the musculoskeletal system such as arthrosis, at each joint to be treated.

IV administration means an administration of the composition or injectable solution directly into the venous system of the subject by means of a catheter or a needle or for example via a pouch of perfusion solute or for example by the tube of the perfuser. Typically, an administration intravenously is implemented in the case of the treatment of thrombocytopenia or arthrosis, or the treatment of a chronic inflammatory bowel disease such as duodenal enteritis or Crohn's disease.

In a particular embodiment, an injectable composition or a solution comprising 1×10⁶ to 1×10⁸ NSCs, in particular 2.5×10⁶ to 1×10⁷ NSCs, more particularly 1×10⁷ NSCs, are administered by intra-articular route, at each joint to be treated, for the treatment of indications of the musculoskeletal system such as osteoarthritis.

In a particular embodiment, an injectable composition or a solution comprising 1×10⁶ to 10×10⁸ NSCs, in particular 1×10⁷ NSCs, is administered intravenously for the treatment of inflammatory diseases involving a problem with the immune system, such as thrombocytopenia.

In a particular embodiment, 1×10⁶ to 5×10⁶ NSC/kg are administered intravenously for treating inflammatory diseases involving a problem with the immune system, such as thrombocytopenia.

In a particular embodiment, an injectable composition or a solution comprising 1×10⁶ to 10×10⁸ NSCs, in particular 1×10⁷ NSCs, is administered intravenously for treating a chronic inflammatory bowel disease.

The administration method is obviously adapted according to the subject and the pathology to be treated. The exact number of cells to be administered depends on various factors, and in particular the age, the weight and the sex of the subject to be treated, the pathology and the extent or severity of the pathology to be treated.

In a particular embodiment, the composition or the injectable solution can be diluted or combined with a pharmaceutically acceptable vehicle before systemic injection intravenously (IV) or more generally parenterally, such as for example a vehicle of the isotonic type, Ringer's lactate associated or not with heparin or 0.9% sodium chloride.

In a particular embodiment, the composition or the injectable solution can be diluted or combined with another curative or palliative therapeutic solution known to persons skilled in the art for the same therapeutic indications as those to which said injectable solution relates.

Another therapeutic solution means the combination with the administration of one or more anti-inflammatory analgesic, immunomodulating, anti-infectious and/or anti-viral treatments related or not to stomatitis.

In a particular embodiment, the pharmaceutical composition or the injectable solution as described previously is used in combination with an anti-inflammatory agent.

This anti-inflammatory agent may be an anti-inflammatory of the steroid or non-steroid type. As steroid anti-inflammatory mention can be made of glucocorticoids or corticosteroids. As non-steroid anti-inflammatory mention can be made of buprenorphine, aspirin, ibuprofen, ketoprofen and methylprednisolone acetate. The anti-inflammatory agent may also be a therapy with an anti-inflammatory action, such as laser therapy.

The analgesic may by way of example be morphinics and alpha2-agonists. The immunomodulator may by way of example be cyclosporines and rapamycin. An anti-infectious and/or antiviral agent may by way of example by antibiotics and recombinant interferons.

The present invention also relates to an in vitro/ex vivo method for manufacturing and preserving a composition comprising a population of NSCs by way of active principle and a cryoprotector, said method comprising the following steps:
a. supplying one or more neonatal biological samples comprising NSCs from one or more individuals,
b. isolating the population of NSCs present in the biological sample or samples,
c. optionally, at least one step of ex vivo amplification of the population of NSCs obtained as step b,
d. formulating the pharmaceutical composition from the population of NSCs obtained at step b or c and of a cryoprotector,
e. packaging the pharmaceutical composition in unit doses,
f. freezing the pharmaceutical composition in unit doses obtained at step e, at a temperature of between −70° C. and −140° C.,
g. optionally, intermediate storage of the pharmaceutical composition at a temperature below −70° C.,
h. final storage of the pharmaceutical composition in unit doses at between −10° C. and −40° C.

In this method, the neonatal biological samples at step a) are as defined previously in the present application. In a particular embodiment, the neonatal biological sample is a placenta and the population of NSCs is therefore a population of placental NSCs.

In a particular embodiment, the population of NSCs comes from a neonatal biological sample from a mammal, and more particularly from a dog, a cat, a horse or a human. In a particular embodiment, the neonatal biological sample is a sample coming from a dog.

“Isolation” in step b), means the operation consisting of extracting via an enzymatic and/or mechanical process, the cells contained in a tissue and the extracellular matrix thereof.

The isolation of the NSC is carried out from a neonatal tissue, for example, by dissection and enzymatic digestion of the tissue, then by centrifugation and recovery of the cell residue containing NSC. Alternatively, it is carried out from a sample of umbilical cord blood. The blood cells can then be separated over a density gradient, in particular by using Ficoll. The cell ring formed at the interphase between the diluted plasma and the Ficoll is recovered, and the cells are washed and centrifuged, then the cell residue containing NSC is recovered. The cells are in general counted and seeded at a density comprised between 10⁵ and 5×10⁵ cells/cm². The total number of cells recovered after centrifugation can be comprised for example between 0.1×10⁶ and 500×10⁶ cellules, more precisely in dogs between 0.1×10⁶ and 10×10⁶, more precisely in horses 100×10⁶ and 500×10⁶.

Following the isolation of the cells containing a fraction of NSC, a step of ex vivo amplification of the NSCs can be carried out by adhesion to the plastic.

In order to obtain more substantial quantities of NSC for the purpose of carrying out different pharmaceutical preparations, this step of amplification of the NSCs can be implemented at various steps of the method.

“Amplification step” means any step that allows for a proliferation of the NSC on a plastic or polymer support.

This phase must be capable of favouring the presence of the NSC to the detriment of other cell types that do not meet the characteristics of the NSC. It must also ensure an optimum proliferation of the cells while still limiting the phenomena of dedifferentiation, of differentiation and/or of senescence. This step involves conditions in a controlled atmosphere such as those skilled in the art are able to establish such as for example with 90% humidity and including 5% CO₂. The amplification temperature must be constant and comprised between 35-40° C., more precisely between 37-39° C. Among the culture media, non-exhaustively, it is possible to mention the mediums of the Alpha-MEM, DMEM, RPMI, IMDM, Opti-MEM, EGM, EGM-2 type, synthetic mediums adapted to the culture of MSC devoid of endotoxin and/or of serum, synthetic media adapted to good manufacturing practices, supplemented or not with foetal bovine serum (FBS) from 0.1% to 20%, platelet lysate, insulin-transferrin-selenium, defined commercial supplements and/or other growth factors and/or molecules that favour the proliferation of NSCs while still limiting the senescence thereof such as FGF, EGF, VEGF, dexamethasone and/or A2P.

This amplification phase can be carried out on different supports once the NSC population is obtained following the isolation step. These different supports can be of a 2D or 3D nature.

Amplification on 2D support means any methods of cell culture allowing for an amplification of the NSCs on single-layer support and correctly developed by those skilled in the art. In particular embodiments the cells can be cultivated in plastic culture dishes treated or not to favour cell adhesion, of the flask type, with one or more stages and/or of the multi-layer type with or without continuous perfusion, with or without optimisation of the air flow. The 2D support can be assimilated to a monolayer culture on a non-flat or curved support such as a cylindrical support of the “Roller Bottles” type (Corning, United States).

Amplification on 3D support means any techniques known by those skilled in the art using biomaterials, porous artificial membranes, microcarriers and/or polymers able to ensure an amplification of the NSCs in a bioreactor and correctly developed by those skilled in the art. In particular embodiments, the NSCs can be amplified in bioreactors under agitation, axial and/or tilting, under wave agitation, under rotating bed agitation, in static and/or infused bioreactors. The biomaterials and/or microcarriers can be of several natures and according to particular embodiments can be of sizes comprised between 100-500 μm in diameter, have a porosity of a different nature, have a treated surface, negatively or positively charged or not, include growth factors or recombinant proteins of the integrin and/or extracellular matrix type or any other biological/chemical molecules that favour cell adhesion and/or cell proliferation.

As the NSC are adherent cells, in order to ensure the amplification step, a cell passage of the NSC can be necessary and carried out by a method correctly developed by those skilled in the art. A cell passage (P) corresponds to the detaching of cells from their support when they arrive at confluence (cell layer), to put them back into culture on a new support. Typically, the detaching of cells can be carried out under the effect of mechanical action, enzymes and/or inhibitors such as, non-exhaustively, trypsin, EDTA and/or recombinant or animal accutase. It is also possible to carry out these cell passages through the use of biomaterials/microcarriers that can be dissolved according to a method developed by those skilled in the art. In the context of culture in a bioreactor and alternatively, the amplification phase may be the subject of a gradual increase in culture support (such as microcarriers, of nutriments/culture media and/or of growth factors. This “fed batch” principle makes it possible to optimise the production of biomass actually within an amplification phase.

In a particular embodiment, for the amplification phase, the cells are for example treated with trypsin-EDTA, then taken in an amplification medium and centrifuged. After taking in the amplification medium, they are put back into culture at a rate of 1,000 to 5,000 cells/cm² or 3D equivalent in amplification medium with or without monitored follow-up of the microenvironment and/or culture atmosphere.

In a particular embodiment, the amplification step can comprise several cell passages.

During this amplification step, the NSCs multiply via cell doubling. Thus, the amplification step can also be defined in terms of cell doubling. In a particular embodiment, said method comprises an amplification step wherein the NSC population according to the invention undergoes 2 to 25 cell doublings, more particularly 5 to 15 cell doublings.

Optionally, after step b. of isolation or step c. of amplification, the population of NSCs can be subjected to a cryopreservation step at −70° C. or −196° C. This corresponds to the creation of an NSC masterbank. “Cryopreservation” means the step of storing frozen cells for a period ranging from 1 day to 5 years and more. The cell bank is conditioned in such a way as to guarantee the integrity and the biological properties of the cell populations.

In a particular embodiment, this freezing step corresponds to a progressive drop in the temperature of the cell suspension (−1° C./minute) to reach the temperature ranging from −70° C. to −196° C. In an alternative embodiment, the cells can be cryopreserved at a freezing speed comprised between −0.3° C./minute and −99° C./minute. The same freezing protocol can comprise one or more different freezing speeds such as in the case of a gradual rising in the freezing speed. In an alternative embodiment, the cells are frozen directly without temperature control in a storage enclosure of which the temperature is comprised between −70° C. and 196° C. The final storage can be in the liquid phase (of the liquid nitrogen type) or gas phase (of the gas phase nitrogen type) or under a controlled temperature condition (of the chamber type −140° C., −100° C., −80° C.). This step affects neither the viability of the cells nor the therapeutic properties thereof.

These cells can next be thawed to undergo an amplification step as described at step c. and/or to be formulated according to step e.

Optionally, stimulation by physical, biological and/or chemical effect of the population of NSCs obtained at step b. or c. or after the above optional cryopreservation step, can be implemented.

The population of NSCs is next formulated at step d. as a pharmaceutical composition. For this purpose, the cells are centrifuged and then taken up in a solution comprising a cryoprotector as described previously in the present application.

In particular, said composition comprises between 0.5 and 30% DMSO, more particularly between 2 and 10%, even more particularly 5%. Alternatively, the cells are taken up in the Cryostor® CS5 solution, a commercial solution comprising 5% DMSO, or any other commercial or preformulated solution containing 5% DMSO.

In a particular embodiment, said composition is of the DMEM medium enriched with 5-50% SVF (vol:vol) and comprising 5 to 10% (vol:vol) DMSO or is a commercial cryopreservation medium containing or not a fraction of DMSO.

The composition is next packaged in unit doses ready for use. The unit doses ready for use may also be termed injectable solutions ready for use.

In particular, the unit dose comprises 1×10⁶ to 1×10⁸ NSC, in particular 1×10⁷ NSCs.

More particularly, the unit dose comprises 1×10⁶ to 1×10⁸ NSCs in a volume of 0.1 ml to 15 ml of composition. In particular, it comprises 1×10⁶ to 5×10⁷ NSCs in a volume of 0.1 ml to 15 ml of composition. In particular, it comprises 2.5×10⁶ to 1×10⁷ NSCs for a volume of 0.1 ml to 15 ml of composition. In particular, it comprises 1×10⁶ to 1×10⁷ cells in 0.5 to 2 ml of composition.

The composition, in the form of unit doses, is next frozen at a temperature between −70° C. and −140° C., in particular between −70° C. and −100° C., more particularly at −80° C. This freezing step is for example implemented under a controlled temperature drop condition at the rate of −1° C./min to a final temperature of between −70° C. and −140° C., using for example a CoolCell® Cell Freezing Containers (BioCision) container or a programmable freezer of the Digitcool (Cryobiosystem) type or a programmable cryogenic freezer of the FREEZAL (biofluid) type.

In a particular embodiment, the cells are preserved at a temperature between −70° C. and −140° C., in particular between −70° C. and −100° C., more particularly at −80° C., for at least 2 h, in particular between 2 h and 36 months, more particularly between 2 h and 24 months.

The composition comprising the population of NSCs thus frozen in the form of unit doses can optionally be subjected to a step f. of intermediate storage, where it is preserved at temperatures below −70° C., in particular between −70° C. and −196° C. for several months, in particular at least 1 day, more particularly between 1 day and 24 months, even more particularly between 1 day and 12 months.

At the end of step f. or of step g. the composition is subjected to a step h. referred to as “final storage”. “Final storage” means storage for a period ranging from 1 day to 5 months, more particularly from 1 day to 3 months, more particularly from 1 day to 1 month of the unit dose, under low temperature conditions.

In particular, this final storage is of at least 21 days, in particular between 21 days and 5 months.

Low temperature conditions means that the preservation temperature for the final storage is between −10° C. and −40° C., more particularly −10° C. and −35° C., more particularly −15° C. and −30° C., even more particularly −17° C. to −20° C., typically of −18° C.

At the end of the final storage, the composition is thawed, and can be directly used in cell therapy. This thawing step is carried out in such a way as to pass from the stage of frozen cells to the stage of thawed cells while limiting cell death via desiccation, mechanical lesion of the plasma membrane, osmotic shock. In a particular embodiment, the composition is heated by manual friction for less than 10 min. In another particular embodiment, the composition is placed in a liquid or dry water bath, adjusted to a temperature comprised between 30 and 40° C. for at least 10 min, in particular at 37° C. for 3 to 5 min. In another particular embodiment, the composition is placed in an automatic thawing apparatus. In another particular embodiment, the composition is thawed at ambient temperature for less than 10 min.

Optionally, after freezing and before administration to the patient, the composition can be put back in suspension in a pharmaceutically acceptable medium for systemic or intravenous injection such as Ringer's lactate or other ionic solution.

In a particular embodiment, said in vitro/ex vivo method for manufacturing and preserving a composition comprising a population of NSCs by way of active principle and a cryoprotector, is an in vitro/ex vivo method for manufacturing and preserving a composition comprising a population of NSCs of phenotype MHC-I^{L}/CD90^{H} by way of active principle and a cryoprotector.

Thus, in a particular embodiment, said in vitro/ex vivo method for manufacturing and preserving a composition comprising a population of NSCs by way of active principle and a cryoprotector comprises, after step b. or c., a step of qualification of the population as being of MHC-I^{L}/CD90^{H} profile as described in the invention.

In a particular embodiment, the present invention also relates to an in vitro/ex vivo method for manufacturing and preserving a composition comprising a population of NSCs by way of active principle and a cryoprotector, said method comprising the following steps:
a. the supply one or more neonatal biological samples comprising NSCs from one or more individuals,
b. isolating the population of NSCs present in the biological sample or samples,
   c. optionally, at least one step of ex vivo amplification of the population of NSCs obtained at step b.,
d. a step of qualifying the population as being of MHC-I^{L}/CD90^{H} profile as described in the invention,
e. formulating the pharmaceutical composition from the population of NSCs obtained at step d. and a cryoprotector,
f. packaging the pharmaceutical composition in unit doses,
g. freezing, at a temperature of between −70° C. and −140° C., the pharmaceutical composition in unit doses obtained at step e.,
h. optionally, intermediate storage of the pharmaceutical composition at a temperature below −70° C.,
i. final storage of the pharmaceutical composition in unit doses at between −10° C. and −40° C.

### BRIEF DESCRIPTION OF THE FIGURES

FIG. 1 shows the procedure for analysing the cell viability by flow cytometry. A first analysis window (FSC-H/SSC-H) is traced so as to take into account only the cells (left-hand graph). The viability of these cells is analysed by measuring the fluorescence of propidium iodide (IP) on channel FL-2 of the cytometer.

FIG. 2 is a graph showing the cell viability evaluated with Trypan Blue (BT) and with propidium iodide (IP) after preservation of the NSCs at −80° C. for 15 days (J15), 1 month (1 M), 2 months (2 M), 3 months (3 M) and 5 months (5 M).

FIG. 3 is a graph showing the percentage of positive cells for expressing the CD29, CD44 and CD90 markers, measured in flow cytometry, after various preservation times for the NSCs at −18° C. (from 15 days to 3 months).

FIG. 4 is a graph showing the proliferative activity of the NSCs through the number of doublings (DN) of the cell population in 7 days, after preservation of the cell population at −80° C. or at −18° C. for 15 days, 1 month, 2 months, 3 months or 5 months.

FIG. 5 is a graph showing the proliferative activity of the NSCs through the doubling time (DT) of the cell population during 7 days, after preservation of the cell population at −80° C. or at −18° C. for 15 days, 1 month, 2 months, 3 months or 5 months.

FIG. 6 is a graph showing the proliferative activity of the NSCs after 24 h, 48 h and 5 days in culture measured by means of the CCK8 colorimetric kit, after preservation of the NSCs at −80° C. or at −18° C. for 15 days, 1 month, 2 months or 3 months.

FIG. 7 is a graph showing the inhibiting activity of the NSCs on the proliferation of lymphocytes (PBMC proliferation ratio), after freezing of the NSCs at −80° C. and preservation at −18° C. for 15 days, 1 month, 2 months or 3 months. The results are compared with the lymphocyte control stimulated with concanavalin A (Con A).

FIG. 8 is a graph showing the inhibiting activity of the NSCs on the proliferation of lymphocytes (PBMC proliferation ratio), after freezing of the NSCs at −80° C. and preservation at −18° C. for 15 days, 1 month, 2 months or 3 months. The results are compared with the inhibiting activity of the NSCs frozen at −80° C. and preserved at −80° C. (−80° C.) for 2 months.

FIG. 9 is a graph showing the cell viability of the NSCs measured by Trypan Blue counting, after a method according to the invention (freezing of the NSCs at −80° C., then preservation at −18° C. for 1 week or 3 months) of after a method comprising freezing of the NSCs at −18° C. and then preservation at −18° C. for 1 week or 3 months.

FIG. 10 is a graph showing the inhibiting activity of the NSCs on the proliferation of lymphocytes (PBMC proliferation ratio), after freezing at −18° C. and preservation at −18° C. of the cell population for 3 months, for co-cultures of ratio 1:10 and 1:20.

FIG. 11 is a graph showing the inhibiting activity of the NSCs on the proliferation of lymphocytes (PBMC proliferation ratio), after freezing of the NSCs at −18° C. and preservation at −18° C. for 3 months, versus freezing of the NSCs at −80° C. and preservation at −18° C. for 3 months, for co-cultures of ratio 1:10.

FIG. 12 shows examples representing various cytometric profiles and the associated characteristics thereof. NSCs are isolated in canine placenta and amplified over several passages (2 to 5). Several samples of NSC are analysed in flow cytometry to evaluate the expression of CD90 (FL2-PE) and of MHC-I (FL4-APC). According to the CD90/CMH-I phenotype profile, the NSCs can be classified in several categories depending on their biological characteristics and their composition. Here the cells do not meet the criteria for expression of CD90/CMH-I, as described in the invention.

FIG. 13 shows examples representing various cytometric profiles and the associated characteristics thereof. NSCs are isolated in canine placenta and amplified over several passages (2 to 5). Several samples of NSC are analysed in flow cytometry to evaluate the expression of CD90 (FL2-PE) and of MHC-I (FL4-APC). According to the CD90/CMH-I phenotype profile, the NSCs can be classified in several categories depending on their biological characteristics and their composition. Here the cells meet the criteria for expression of CD90/CMH-I, as described in the invention.

FIG. 14 shows the viability of the thawing of a population of cells frozen at −80° C. and then preserved at −20° C. over two months (on the right), according to its composition in terms of phenotype MHC-I^{L}/CD90^{H} cells (on the left).

### EXAMPLES

l. Isolation of the NSCs from Canine Placenta

The canine extra-embryonic annexes (placenta, umbilical cord) are aseptically sampled during caesareans practiced in gestating dogs at term. As soon as the new-born puppy is removed from the amniotic sac and placed in safety, the extra-embryonic tissue is immediately transferred to a transport box containing a buffered saline solution with Dulbecco's phosphate (D-PBS) to be transported to the laboratory. The treatment of the extra-embryonic tissue is carried out at most within 48 h following sampling. All of the treatment steps of the tissue are carried out in a controlled environment, under a biosafety cabinet (BSC).

The tissue is transferred to a 100 cm² Petri dish and the residual amniotic membrane is mechanically removed by dissection. The placenta is placed embryonic face against the plastic surface of the box and the uteroverdin present on the face of maternal origin is separated from the placenta by scraping the tissue. The placenta is rinsed 3 to 5 times in successive baths of D-PBS. The blood vessels and the umbilical cord are then mechanically removed from the placenta. The placental tissue is dissected into fragments of about 10-20 mm² then subjected to an enzymatic digestion by incubating the tissue fragments in a solution composed of DMEM (Dulbecco's modified Eagle medium) containing 0.5-4 mg/ml of type I collagenase, and more specifically a concentration of 1 mg/ml of type I collagenase. The enzymatic digestion takes place at 37° C. for 1 h but a digestion comprised between 30 min and 16 h can be carried out by decreasing the incubation temperature (ambient temperature (18-22° C. or 4° C.). At the end of the digestion, the enzymatic activity is stopped by dilution, by adding DMEM containing at least 10% foetal bovine serum (FBS) in a quantity equivalent to the solution of enzymatic digestion. The solution is then filtered over a 70-100 μm screen. The recovered cells are centrifuged at 800 g for 10 min. The cell residue containing the neonatal stromal cells is rinsed with DMEM and again centrifuged at 800 g for 10 min. The cell residue is taken in culture medium constituted of DMEM, 10% FBS, 2 mM glutamine and from 0 to 20 ng/ml of fibroblast growth factor (FGF). The cells are counted and seeded in culture dishes at a density comprised between 10⁴ and 2.10⁴ cells/cm². The cells are then cultivated in the culture medium described hereinabove in a controlled atmosphere at 37° C. and containing 5% CO₂. The medium is changed after 48 h then every 2-3 days. The cells are passed when the confluence reaches 70-80%.

II. Cell Passage and Amplification

At sub-confluence, the cells undergo a cell passage and optionally an amplification procedure. The NSCs are rinsed with D-PBS and treated with 0.05% trypsin-EDTA for 2-5 min at 37° C. This makes it possible to detach the cells and to form a population of isolated cells. The cells are then taken with amplification medium constituted of DMEM, 10% FBS, 2 mM glutamine and from 0 to 20 ng/ml of fibroblast growth factor (FGF) and centrifuged between 300-500 g for 5 to 10 min. The NSCs are taken in amplification medium, and counted via manual counting (Trypan Blue and Malassez cells) or using a cytometer. They are then seeded for 1,500 to 5,000 cells/cm² and cultivated on a plastic cell culture support in an amplification medium and in a controlled atmosphere at 37° C. and containing 5% CO₂. During the amplification process the cells can undergo between 0 and 15 cell passages.

III. Optional Cryopreservation of the NSCs—Formation of a Masterbank

At the end of the first or second cell passage (P1-P2), the cells can be cryopreserved in seed units. To do this, after counting, the NSCs are centrifuged between 300-500 g for 5 to 10 min and the cell residue is taken in the freezing medium comprised either of DMEM medium enriched with 5-20% FBS and 5-10% (vol:vol) DMSO or in a commercial cryopreservation medium, containing or not a fraction of DMSO. The cell concentration is comprised between 1.10⁶ and 15.10⁶ cells per ml of freezing medium. The freezing of the cells is carried out in controlled falling temperature conditions, using for example a CoolCell® Cell Freezing Container (BioCision) and by following the freezing procedure as described by the manufacturer. The cells are then transferred for negative cold storage at temperatures comprised between −70° C. and −196° C.

The cell units can be used to generate cell units for therapeutic use. The seed units are thawed at 37° C. for 3-6 min and amplified in vitro. The cells are seeded in the culture medium at the density of 1,500-3,000 cells/cm2. The cells are amplified by successive passage in vitro. When a significant number of cells are produced (for example >150.10⁶cells), the cells are frozen according to the protocol described hereinabove. The cells are distributed into hermetically sealed bottles with seals at a rate of 1.10⁶-15.10⁶ cells/ml in a freezing medium free from product of animal origin (such as for example the cryopreservation medium Recovery™ Cell culture freezing medium (Thermo Fisher), or Cryostem™ freezing medium (Biological Industries). The bottles are lowered in temperature according to a controlled falling temperature protocol, at a rate of −1° C./min to −80° C. The bottles are then transferred at −80° C. for storage. Once obtained, the population of NSCs is characterised on the one hand by its structural characteristics (presence/absence of markers) and on the other hand by its functional characteristics (proliferation, differentiation etc.).

IV. Preparation of the Pharmaceutical Composition and Freezing of the Unit Doses

At the end of the amplification of the NSCs corresponding to a phase of production of the unit doses for therapeutic purposes, the cells can be frozen in the form of a pharmaceutical composition and packaged in unit doses. To do this, after counting, the NSCs are centrifuged at between 300 and 500 g for 5 to 10 min and the cell remainder is taken up in freezing medium free from any product of animal origin composed of Cryostor® CS5 medium (stem cell technology) or any other commercial or preformulated medium containing 5% DMSO. The cells are distributed in hermetically sealed cappable bottles at the rate of 1×10⁶-15×10⁶ cellules/ml. The cells are frozen in a controlled temperature drop condition, using for example a programmable cryogenic freezer of the FREEZAL type (biofluid) and programmed for a temperature drop of 1° C./min for an initial temperature of between 10° C. and 4° C. and for a final temperature of −80° C. The cells are then transferred for negative cold storage at temperatures of between −70° C. and −80° C. or directly stored for final storage at a temperature of between −10° C. and −40° C.

V. Details and Samples Tested: Freezing/Storage Comparison −80° C./−18° C. versus −80° C./−80° C.

Three batches of CSN coming from canine placentas of various amplification phases are used in this study.

Pharmaceutical compositions as described in the invention, comprising 15×10⁶ NSC in 1.5 ml of Cryostor® CS5 medium (stem cell technology), a medium comprising 5% DMSO, were packaged in bottles and were cryopreserved at −80° C. at the rate of a temperature drop of −1° C./minute using the Coolcell device. Three bottles in each batch were stored at −80° C. for a period of between 15 days and 3 months (Table 1), and then transferred to the freezer at −18° C. (condition −80° C./−18° C.).

[Table 1]

TABLE 1

| | Cell | Storage times −80 for | | |
|---|---|---|---|---|
| | passage on | analysis of intermediate | Thawing | Thawing |
| Batch | freezing | viability | viability (TB) | viability (IP) |
| 1 | P3 | 3 months | 94.2% | 89.6% |
| 2 | P5 | 2 months | 90.3% | 85% |
| 3 | P5 | 15 days | 90.2% | ND |

In parallel, cells stored only at −80° C., i.e. without transfer to −18° C., were used as controls (condition “ctrl −80° C.”).

On the day of the analyses, the bottles are taken out of −18° C. or −80° C., transferred to the laboratory in a polystyrene container containing a −20° C. eutectic pack. The bottles are thawed to ambient temperature (18-25° C.) for approximately 5 minutes. The cells are sucked from the bottles by means of a syringe (18 G needle) and transferred into a sterile Eppendorf for the following analyses.

V.1. Viability Under Trypan Blue and Propidium Iodide

For each condition, an aliquot of the initial suspension (50 μl) is sampled for counting with Trypan Blue (TB) using a Luna electronic counter.

Another aliquot of the initial suspension (50 μl) is sampled for marking with flow cytometer with propidium iodide (IP) (Sigma Aldrich). The aliquot is kept in a refrigerator until analysis.

The PI analysis window of the cytometric analysis (BD accury c6) is traced in accordance with the following figure. A “cell” window is traced in FSC-H/SSC-H and is used for the PI analysis. The PI analysis is carried out on the FL-2 channel by an FL2-A/FSC-H analysis (FIG. 1).

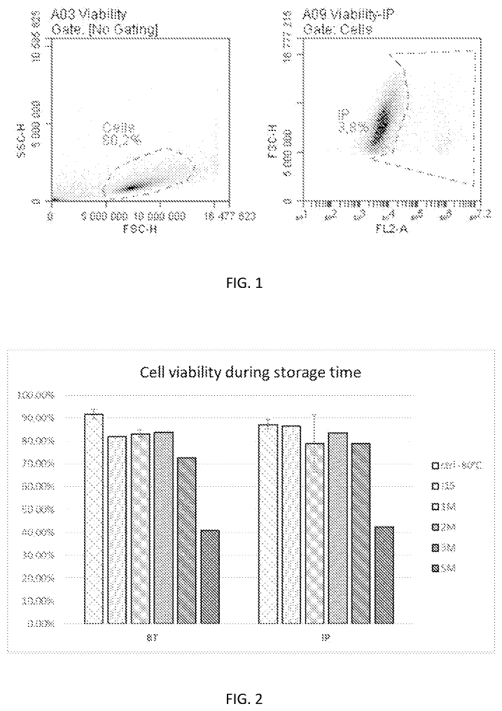

The cell viability was evaluated by TB and PI for all the conditions tested (15 days: J15 n=2; 1 month: 1 M n=3; 2 months: 2 M n=2; 3 months: 3 M n=1; 5 months: 5 M n=2) and compared with the viability of the cells after thawing of the cell batches stored at −80° C. (n=3) (FIG. 2).

The results obtained after the storage time at −80° C. for intermediate viability analysis (Table 1) did not show any differences compared with the cells frozen and stored in accordance with the protocol of the present invention (freezing at −80° C. and then storage at −18° C.) (FIG. 2).

The results obtained by TB or PI tally, suggesting that the two analytical methods can be used for verifying the viability parameter. The data reveal a viability >80% for the samples stored up to 2 months at −18° C. in TB and PI (greater variability at 1 month in PI). Analysis at 3 months shows a viability of 72% (TB) and 79% in PI. The analyses after 5 months at −18° C. show a mean viability of 40% (TB) and 42% (PI). The results show that the NSCs can be stored for a period of at least 3 months at −18° C. without drastically affecting their viability on thawing.

V.2. Analysis of Phenotype Markers

Cytometric analysis aims to determine the presence of membrane markers on the surface of the NSCs by means of the use of a panel of antibodies specific to each marker, for example: CD29, CD44, CD90.

In order to study the impact of storage at −18° C. on the frozen cells, 5×10⁵ cells are sampled from each previously mentioned condition for cytometric analysis. Each sampling of each condition is separated into 4 samples transferred into 1.5 ml Eppendorfs in order to implement 4 markings. The cells are taken up in 1 ml of cytometric buffer composed of D-PBS and 0.5-1% (v/v) bovine serum albumin (BSA) or

0.5-2% (v/v) foetal bovine serum. These 4 samples are centrifuged at 500 g for 5 min. A second washing is carried out under the same experimental conditions. After elimination of the supernatant, the cells of the 4 tubes are respectively taken up in 1 volume of 30-100 μl, in particular 50 μl, of cytometric buffer. In total, 4 markings are implemented per freezing and storage condition: isotypic marking (FITC and PE), CD29 marking, CD44 marking and CD90 marking in accordance with Table 2.

[Table 2]

TABLE 2

| Mark | Fc | Reference | Name of product | Supplier | TypeAc | Clone |
|---|---|---|---|---|---|---|
| Isotype | FITC | 400110 | FITC mouse IgG1 k isotype | Ozyme | IgG1k | MOPC-21 |
| | | | Ctrl_Fc | | | |
| Isotype | PE | 400114 | PE mouse IgG1 k isotype | Ozyme | IgG1k | MOPC-21 |
| Isotype_primary | — | COL2002 | Mouse COL2002/COLIS205C | Vetmed | IgG2a | COLIS205C |
| | | | IgG2a | | | |
| Secondary | APC | 17-4010-82 | Goat F(ab′) 2 anti-mouse IgG | Thermofisher | IgG | Polyclonal |
| | | | secondary a pc | | | |
| CD29 | PE | 303003 | PE anti-human CD29 | Ozyme | IgG1k | TS2/16 |
| CD44 | APC | 103012 | APC rat anti-human CD44 | Ozyme | IgG2bk | IM7 |
| CD90 | PE | 12-5900-41 | PE anti-canine CD90 | Thermofisher | IgG2bk | YK IC337,217 |
| CMH1 | — | DG-BOV2001 | Primary anti-canine CMH-1 | Vetmed | IgG2a | DG-H58A |

The optimum concentration of antibodies used for the marking must be determined beforehand by those skilled in the art. The incubation required for the marking must also be determined by those skilled in the art and comprised between 15 min and 10 h at 4° C. in a dark place. In particular, the cells are incubated 20 min at 4° C. in a dark place.

Isotypic controls (here FITC, PE and APC for fluorescene isothiocyanate, phycoerythrin and allophycocyanin) adapted to each marking have to be used as a negative control. Following the incubation with the antibodies, the cells are washed with D-PBS, centrifuged 5-10 min at 500 g and taken in a volume from 100 to 250 μI of marking buffer for analysis with the flow cytometer (Accuri C6, BD Biosciences).

Thus the expression of the CD29, CD44 and CD90 markers was evaluated by flow cytometry. The mean expression of the markers of the cells stored at −80° C. at variable times was taken as control (CD29=91%±7%; CD44=99.4%±0.3%; CD90=87.8±9%) (n=6).

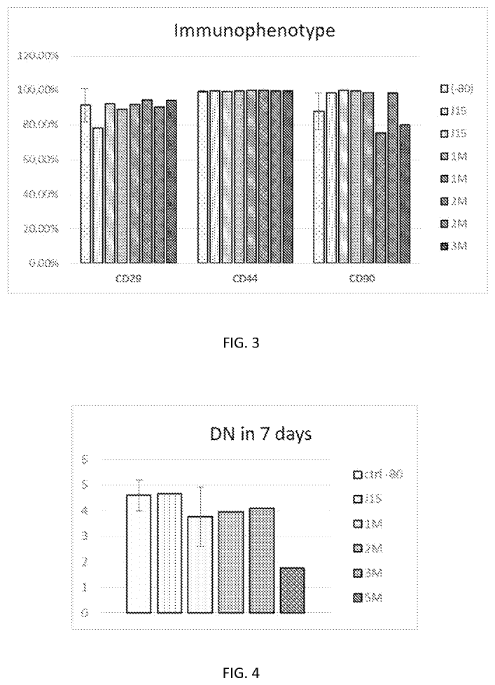

The samples were analysed for 15 days: J15 n=2; 1 month: 1M n=3; 2 months: 2M n=2; 3 months: 3M n=1. For the 3 markers studied, a positive signal was revealed whatever the condition tested (FIG. 3).

FIG. 3 representing the percentage positive cells for the CD29, CD44 and CD90 markers. The results show a very similar expression of CD44 for all the conditions (>99% positivity). With regard to CD29, >85% positivity is observed for all the conditions (except a sample stored at −18° C. for 15 days). The CD90 marker shows a greater variability with 5 samples preserved at −18° C. the positive signal of which is >95% and two samples with a positive signal of approximately 80%. This variability is also observed with samples preserved at −80° C. (87.8±9%) and the samples in the course of amplification. Because of this, preservation at −18° C. does not affect the expression of the CD29, CD44 or CD90 markers.

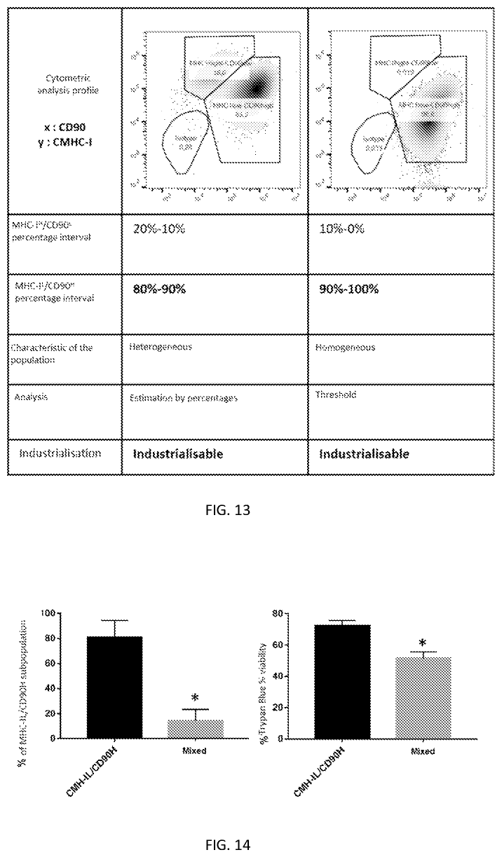

For all the experiments relating to the pharmaceutical composition described in the invention, the MHC-1^{L}/CD90^{H} profile is determined in advance during the cell amplification. The profile is determined by flow cytometry analysis and immunomarking of the MHC-I and CD90. The details of the analysis are presented in the invention (paragraphs [0065] to [0104]). The profile is determined in accordance with a cytometric analysis matrix presented in FIG. 13. The profile is termed MHC-I^{L}/CD90^{H} if the MHC-1^{L}/CD90^{H} sub-population represents more than 80% of the total population at P5.

V.3. Proliferative and Metabolic Activity After Culture of the Thawed Cells

After thawing of the pharmaceutical composition and counting, the cells are diluted in amplification medium (DMEM, 10% SVF, 2 mM glutamine and 0 to 20 ng/ml of FGF) to obtain a concentration of approximately 10⁶ cells/ml. A 75 cm² culture dish is seeded with 0.225×10⁶ cells for proliferation analysis for 1 week in amplification medium. In parallel, a 96 well plate is seeded to the extent of 10,000 cells/well for kinetic monitoring of the metabolism of the cells using Cell Counting Kit-8 (CCK8-Sigma-Aldrich) analysed by spectrophotometry.

The proliferative activity of the NSCs is evaluated by calculating the number of doublings (DN) of the cells during 7 days of culture and calculating the doubling time (DT). After 7 days of culture, the cells are detached from their culture substrate by means of trypsin/EDTA 0.5% for 2-3 minutes. Culture medium is added and the cells are centrifuged 5 min/300 g. The cell residue is returned to a defined volume of culture medium and the cells are counted by a Trypan Blue exclusion technique by means of an electronic counter. The number of doublings (DN) is calculated with each cell passage according to the following formula: DN=LOG (Nf/Ni)/LOG(2) (Nf: number of final cells and Ni: number of initial cells). The doubling time (DT) is calculated in accordance with the following formula: DT=culture time (h)/DN.

FIGS. 4 and 5 present the proliferative activity of the cells in culture for 7 days. FIG. 4 presents the number of doublings of NSCs cultivated for 7 days and FIG. 5 the mean doubling time of the cultures. The data show a mean number of cell doublings (DN)=4.6 when the cells have come from −80° C. (n=12). This number is similar for the two samples stored at −18° C./15 days (DN=4.6). After 1 month at −18° C., ND=3.6±1.2 (n=3); after 2 months at −18° C., ND=3.9 (n=2) and after 3 months at −18° C., ND=4.1 (n=1). A significant reduction in the number of doublings is observed after 5 months at −18° C.; ND=1.7 (n=2) (FIG. 4).

With regard to the doubling times (DT), these are similar to −80° C. and after 15 days at −18° C.; respectively 37.5 h±5 (n=12) and 38.7 h (n=2). At 1, 2 and 3 months at −18° C., respective doubling times are observed of 48.5 h±18 (n=3), 48.7 h (n=2), and 41 h (n=1). After 5 months at −18° C., the doubling times increase substantially DT=112 h (n=2) (FIG. 5).

V.4. Metabolic Activity

A second analytical method was used for evaluating the proliferation of the post-thawing cells in 24 h, 48 h and 5 day kinetics as well as for checking the metabolic increase in the cells during culture. The CCK8 colorimetric test was used and analysed in plate reading at 450 nm.

The analyses were done on cells preserved at −18° C. for 15 days (n=2); 1 month (n=2); 2 months (n=2); 3 months (n=2) and 4 samples cryopreserved at −80° C. used as controls.

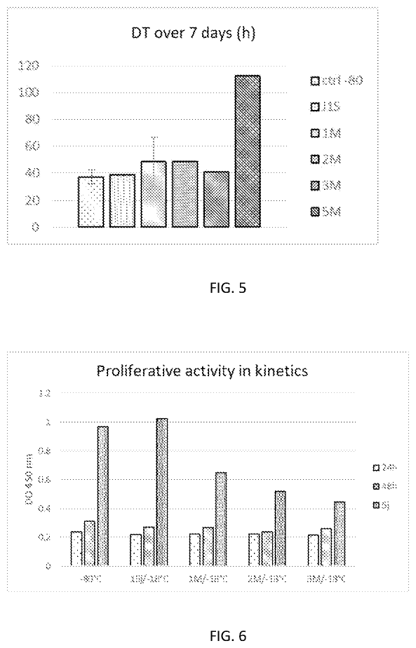

FIG. 6 presents the analysis of the proliferative activity of the cells after 24 h, 48 h and 5 days in culture calculated by means of the CCK8 colorimetric kit. An increase in the OD corresponds to an increase in the metabolic activity of the cells or in the number of viable cells in the wells.

The results show that, at 24 h post-seeding, the optical densities (ODs) at 450 nm are equivalent for all the conditions (OD₄₅₀=0.22). This is consistent with the viability results obtained on thawing.

At 48 h, an increase in OD is observed in the −80° C. condition (OD₄₅₀=0.313±0.1) whereas the ODs are not modified in the −18° C. tests (OD₄₅₀ (J15)=0.27; OD₄₅₀ (1 M)=0.27; OD₄₅₀ (2 M)=0.24; OD₄₅₀ (3 M)=0.26).

V.5. In Vitro Immunosuppressive Activity of the NSCs

The immunomodulating activity of the NSCs coming from the pharmaceutical composition is studied through their immunosuppressive activity, in particular their ability to inhibit proliferation of the lymphocytes in vitro.

This inhibiting activity is evaluated by co-cultivating the NSCs of each storage condition with blood mononuclear cells (PBMCs) in the presence of a mitogenic agent.

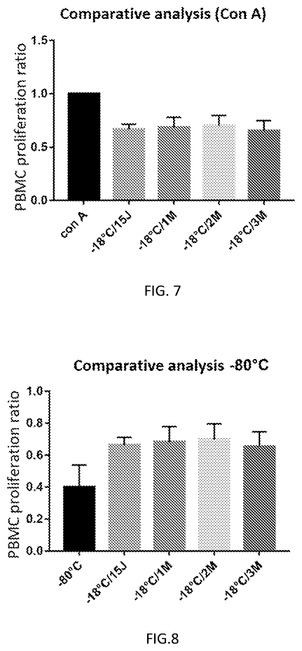

To achieve this, the PBMCs are isolated from a blood sample taken from a donor dog or a donor horse over a Ficoll gradient. The PBMCs are then incubated with a fluorescent dye (CellTrace CFSE, Thermo Fisher) which makes it possible to measure cellular proliferation over several generations. 0.2.10⁶ PBMCs marked by Celltrace are added to the wells of a 96 well plate in which the NSCs were seeded the day before in a concentration (2.10⁴ NSC/wells); thus making it possible to obtain a ratio of NSC:PBMC equivalent to 1:10. The NSCs are treated with mitomycin (10 μg/ml for 1.5-2 h at 37° C.) and rinsed 3 times in culture medium before the addition of PBMCs. The proliferation medium of the lymphocytes is added (RPMI, 10% SVF, 2 mM glutamine, 10 mM hepes, 50 μM β-mercaptoethanol, 5 μg/ml concanavalin A). The PBMC/NSC co-cultures and the control cultures (PBMC without NSCs) are incubated for 4 days in an incubator at 37° C. Following the culture, the non-adherent cells are recovered from the wells, centrifuged and washed in D-PBS. The cells are then marked with an anti-CD3 antibody coupled with a FITC fluorochrome for 30 min at 4° C. The cells are then washed in D-PBS before flow cytometry analysis (Accuri C6, BD Biosciences). The cytometric analysis consists of evaluating the Celltrace signal within the viable CD3+ population. At the end of incubation, the T-lymphocytes specifically marked with CD3 are analysed by cytometer. For each test implemented, the PBMCs were isolated from 2 or 3 patients. A lymphocyte proliferation index is calculated for each experimental condition using the Modfit® analysis software. The lymphocyte proliferation index in the presence of NSCs is standardised with respect to the lymphocyte proliferation index under controlled condition, fixed at 1 (100% proliferation). This ratio is termed “PBMC proliferation ratio”. The statistical analyses were implemented by a t-test of comparison with the reference value (con A). The conditions stored at −18° C. were compared with the PNMCs stimulated with concavanalin A (con A) (FIG. 7) or with the results of samples stored at −80° C. (−80) (FIG. 8).

The data show a reduction in the lymphocyte proliferation in the presence of NSCs stored for 15 d, 1 M, 2 M and 3 M at −18° C. This difference is significant (p<0.05) with the control condition con A with the cells stored for 15 d, 1 M, 2 M and 3 M at −18° C. (FIG. 7).

The data obtained in this study are compared with the results observed when the PBMCs are co-cultivated with the NSCs stored at −80° C. (n=14), showing a proliferative activity=0.4±0.13 (FIG. 8). The results show a significant difference in lympho-inhibiting activity of the NSCs stored at −18° C. for 15 days, 1 month, 2 months and 3 months compared with the NSCs stored at −80° C. (t-test).

VI. Freezing/Storage Comparison: −80° C./−18° C. versus −18° C./−18° C.

In order to assess whether the cryopreservation mode of the NSCs with a 1^{st} step at −80° C. is necessary for preserving the biological properties of the cells stored temporarily at −18° C., the following study is carried out by storing samples of NSCs in a cryopreservation medium directly in a freezer at −18° C.±5° C. without an initial temperature drop to −80° C.

Briefly, the NSCs are centrifuged at 300-500 g for 5 to 10 min and the cell residue is taken up in freezing medium composed either of DMEM medium enriched with 5-20% FBS and 5-10% (vol:vol) DMSO or in a commercial cryopreservation medium, containing or not a fraction of DMSO. The cell concentration is between 5×10⁶ and 10×10⁶ cells per ml of freezing medium. Some of the samples coming from 5 cell batches are frozen and preserved according to the description of the invention presented in part V.1 (−80/−18). Others are frozen and stored directly at −18° C. (−18/−18). The number of experimental samples per analysis time is presented in Table 3.

[Table 3]

TABLE 3

| Number of doses | −80/−18 | −18/18 |
|---|---|---|
| T0 | 3 | 2 |
| 1 week | 2 | 2 |
| 2-3M | 5 | 4 |

VI.1. Freezing/Storage Comparison: Viability Under Trypan Blue

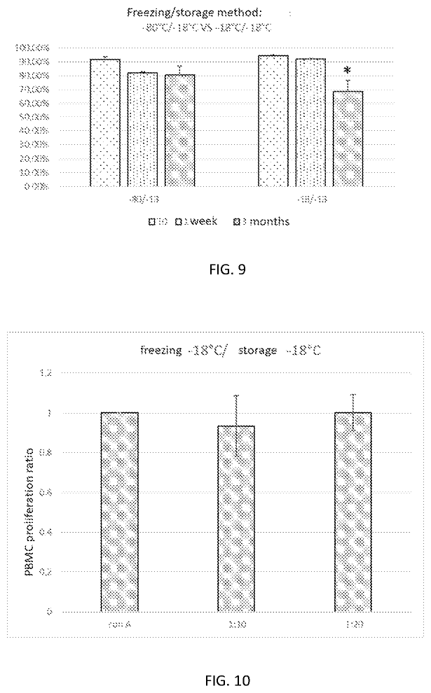

The results in FIG. 9 show that the cell viability is only slightly affected by the two freezing conditions for storage of one week. A reduction of 9.42% is observed for −80/−18 and 2.20% for −18/−18 at 1 week in comparison with their respective viabilities on freezing. Nevertheless, after a storage of 2-3 months, a significant reduction in the viability can be observed for the −18/−18 case (reduction of 26.01% with respect to T0) while in the case −80/−18, a maintenance of viability is found (reduction of only 11.05% with respect to T0). The statistical analysis at 2-3 months indicates a significant difference in viability between the two methods of the order of 12.03% (comparison of the means per Student's T-test, p=0.041). Because of this, the freezing strategy described in the invention (−80/−18) favours storage of the NSCs at −18° C. for a period of 2-3 months in comparison with the −18/−18 method.

VI.2. Immunomodulator Effect of the NSCs According to the Freezing/Storage Method −18° C./−18° C.

The immunomodulating activity of the NSCs was studied through their immunosuppressive activity, in particular their ability to inhibit the proliferation of the lymphocytes in vitro as detailed in part V.5.

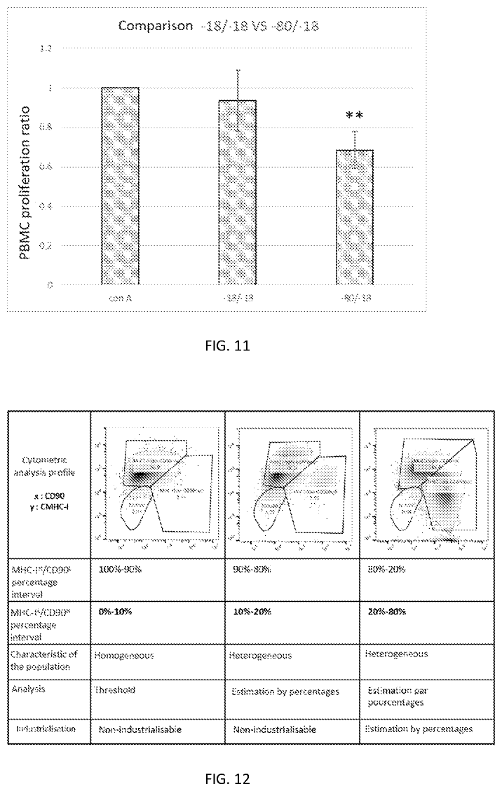

The NSCs frozen at −18 and stored at −18° C. for 3 months were thawed and cultivated with PBMCs marked with CFSE. The lymphocyte proliferation index in the presence of NSC is standardised with respect to the lymphocyte proliferation index under controlled condition, fixed at 1 (100%). This ratio is termed the PBMC proliferation ratio. The results in FIG. 10 showed that, for co-cultures with an NSC:PBMC ratio of 1:10 or 1:20, no immunomodulating effect could be observed, indicating a loss of this effect following freezing at −18° C. and storage at −18° C. These results were compared with the methods of freezing at −80° C. and storage at −18° C. described in the invention (FIG. 11). The comparison showed that the method described in the invention allowed preservation of the lympho-inhibiting activity of the NSCs compared with a direct freezing at −18° C. (comparisons of the means according to the two techniques by Student's T-test, p=0.0017). In other words, in FIG. 11, the lower the bar, the more the lymphocyte proliferation is inhibited.

VII. Effect of the Proportion of MHC-I^{L}/CD90^{H} Sub-Population on the Viability of the −80° C./−18° C. Product on Thawing

Several expression profiles of the MHC-I and CD90 markers were identified among the populations of NSC coming from canine placenta. Six MSC populations were analysed during the manufacturing method: 2 to 7 analyses for passages of between P3 and P6. These analyses were implemented in accordance with the description of the invention. The expression profiles were determined by flow cytometry analysis and immunomarking of the MHC-I and CD90. A cytometric analysis matrix is established in accordance with FIGS. 12 and 13.

The MHC-I^{L}/CD90^{H} sub-population of interest was quantified and expressed in terms of percentage of the population. A cytometric profile analysis by double marking is implemented in accordance with the description of the invention. Two profiles emerged during these experiments, a profile termed MHC-I^{L}/CD90^{H}, which has at P5 more than 80% of the MHC-I^{L}/CD90^{H} sub-population (n=3). This profile corresponds to that of the pharmaceutical composition described in the invention. This profile is illustrated by FIG. 13. As well as a so-called “mixed” profile wherein the presence of the MHC-I^{L}/CD90^{H} sub-population remained below 80% during the manufacturing method (n=3). This profile does not correspond to that of the pharmaceutical composition described in the invention. This profile is illustrated by FIG. 12. For each NSC population, the percentage mean of the MHC-I^{L}/CD90^{H} population is calculated between the P3 to P6 passages. These results are presented in FIG. 14 (left). The results confirm a predominance of the MHC-I^{L}/CD90^{H} population on these passages for the populations termed MHC-I^{L}/CD90^{H} (comparison of the means by Student's T-test, p=0.0022).

Freezing and storage of these NSC populations are implemented in accordance with the description of the invention (i.e. freezing at −80° C. and storage at −18° C.). These cell products are preserved for 2 months at a temperature of −18° C. They are then thawed and a Trypan Blue viability study is implemented. The data are presented in FIG. 14 (right). The result show that the cells termed MHC-I^{L}/CD90^{H} have viability on thawing superior to the NSCs termed mixed (comparison of the means by Student's T-test, p=0.0135).

### LIST OF DOCUMENTS CITED

For information, the following documents are cited:
Marquez-Curtis, L. A., Janowska-Wieczorek, A., McGann, L. E., and Elliott, J. A. W. (2015). Mesenchymal stromal cells derived from various tissues: Biological, clinical and cryopreservation aspects. Cryobiology 71, 181-197.
Espina, M., Jülke, H., Brehm, W., Ribitsch, I., Winter, K., and Delling, U. (2016). Evaluation of transport conditions for autologous bone marrow-derived mesenchymal stromal cells for therapeutic application in horses. PeerJ 4, e1773.
Chinese patent application CN102487939 A, filed by Shangai Angelife Biotechnology Co Ltd, published on 13 Jun. 2012. Inventors: Yingbo, C., Jiawei, Z., Limin, Y., and Yiqiang, J.
Freshney, R. Ian. (2015). Culture of animal cells: a manual of basic technique and specialized applications. John Wiley & Sons, (p 307-p 312)
Mazur, P. (1984). Freezing of living cells: mechanisms and implications. Am J Physiol 247, C125-142
Gao, D., et al. (1997). Fundamental cryobiology of mammalian spermatozoa, p. 263-328. In A. M. J. Karow and J. K. Critser (ed.), Reproductive tissue banking. Academic Press, San Diego, Calif.

1. Method for freezing and preserving neonatal stromal cells (NSCs) comprising
1) preparing a composition comprising a population of neonatal stromal cells (NSCs) and a cryoprotector;
2) freezing said composition at a temperature of between −70° C. and −140° C.; then
3) storing said composition at a temperature of between −10° C. and −40° C.

2. The method according to claim 1, wherein the population of NSCs is a population of placental NSCs.

3. The method according to claim 1, wherein step 2 is conducted for at least 2 h.

4. The method according to claim 1, wherein step 3 is conducted for 1 day to 5 months.

5. The method according to claim 1, wherein step 3 is conducted for at least 21 days.

6. The method according to claim 1, wherein the cryoprotector is selected from glycerol, dimethyl sulfoxide (DMSO), propylene glycol, proteoglycans, trehalose, polyethylene glycol (PEG), polyacrylic acid, poly-L-lysine, ethylene glycol or a combination of a plurality of these cryoprotectors.

7. The method according to claim 1, wherein the composition comprises DMSO as the cryoprotector.

8. The method according to claim 1, wherein said composition comprises a population of NSCs of phenotype MHC-IL/CD90H.

9. Pharmaceutical composition comprising a population of NSCs and a cryoprotector, wherein the pharmaceutical composition has been preserved at between −10° C. and −40° C.

10. Pharmaceutical composition comprising a population of NSCs according to claim 9, wherein said NSCs are placental NSCs.

11. Pharmaceutical composition comprising a population of NSCs according to claim 9, wherein said population of NSCs is a population of NSCs of phenotype MHC-IL/CD90H.

12. Pharmaceutical composition comprising a population of NSCs according to claim 9, wherein said population of NSCs is free from NSC of phenotype MHC-IH/CD90L.

13. Pharmaceutical composition according to claim 9, wherein the cryoprotector is DMSO.

14. The method of claim 1, wherein the step of freezing is performed at a temperature of between −70° C. and −100° C.

15. The method of claim 14, wherein the step of freezing is performed at a temperature of −80° C.

16. The pharmaceutical composition of claim 9, wherein the pharmaceutical composition has been preserved at a temperature between −10° C. and −35° C.

17. The pharmaceutical composition of claim 16, wherein the pharmaceutical composition has been preserved at a temperature between −15° C. and −30° C.

18. The pharmaceutical composition of claim 17, wherein the pharmaceutical composition has been preserved at a temperature between −17° C. and −20° C.

19. The pharmaceutical composition of claim 18, wherein the pharmaceutical composition has been preserved at a temperature of −18° C.
